(19) United States
(12) Patent Application Publication
(10) Pub. No.: US 2008/0224716 A1
(43) Pub. Date: Sep. 18, 2008

(54) Method and System For Determining Freshness and Palatability and Assessing Organ Vitality

(76) Inventors: Michaeal G. Singer, Harrisville, MI (US); John D. Kutzko, Nokomis, FL (US)

Correspondence Address: WEINER & BURT, P.C., 635 N US-23, POB 186, HARRISVILLE, MI, 48740, US

(21) Appl. No.: 11/912,887

(22) PCT Filed: Feb. 28, 2007
(86) PCT No.: PCT/US07/05164

§ 371 Date: Oct. 27, 2007

Related U.S. Application Data
(60) Provisional application No. 60/826,774, filed on Sep. 25, 2006.
(60) Provisional application No. 60/827,698, filed on Sep. 30, 2006.

(30) Foreign Application Priority Data
Mar. 18, 2006 (US) 11/386016
Oct. 10, 2006 (US) 11/548 003

(51) Int. Cl. G01N 33/02; G01N 27/02

(52) U.S. Cl. 324/629; 426/231; 702/65

(57) ABSTRACT

A method and system to determine freshness and palatability (tenderness, juiciness, and flavor) of live foodstuffs (meat, fish, fowl, fruit and vegetables) including the steps of; utilizing bioelectrical impedance analysis in a biological subject model for measurement and composition analysis; and a system of using the results of the utilizing step procedure to illustrate an objective scale of palatability; a ‘Palatability Index’. Also a method of whole body and regional organ and tissue vitality assessment in a biological entity, human, animal, fruit or vegetable, including the steps of: utilizing bioelectric impedance analysis in a biological model for composition analysis; and using the results of the utilizing step to provide an objective assessment of volume and distribution of fluid and tissues, and electrical health of cells and membranes of the organ or tissue.

The invention relates to methods and systems of determining freshness and palatability of a foodstuff biological entity including at least a portion of a live or previously-live organism, and to methods and apparatus for use in the in vitro and in vivo assessment of organ vitality.

### BRIEF DESCRIPTION OF THE DRAWINGS

FIG. 1 is a schematic illustration of one embodiment of the present invention.

FIG. 2 illustrates how electrodes may be placed on a hand for the BIA testing procedure.

FIG. 3 illustrates how the electrodes may be placed on the foot for the BIA testing.

FIG. 4 illustrates the testing methods for various portions of the body, to indicate where impedance plethysmography diagnostics fits in the testing regimen.

### DETAILED DESCRIPTION OF THE INVENTION

For a first major aspect of the invention, the following terminology applies.

The terms “biological entity”, “patient” and “subject” mean any and all human beings, animals and/or living organisms.

The term “non-acute death” means any death that does not occur acutely; it occurs more than four days (96 hours) from a precipitous event or illness; it is the end-point of a process whose duration exceeds the four-day reference; unlike that death resulting from a proximate, immediate or acute event, a ‘non-acute death’ occurs over time.

BIA (bioelectrical impedance analysis) is an electrodiagnostic methodology based upon the conductive properties of the body's tissues, cells, and fluids. The BIA instrument, such as that disclosed in U.S. Pat. No. 5,372,141, an impedance plethysmograph (IPG), may use a constant current source producing a low-voltage electrical signal, usually 800 micro-amps at a high frequency, often fixed at 50 KHz, although a range of frequencies, electrode arrays and sampling rates may be used to set up an electrical field in the whole body or a body segment using two pairs of surface ECG-type or otherwise configured electrode arrays; on, in or around the body, region or segment.

The invention can utilize a modification of the body composition analyzer disclosed in U.S. Pat. No. 5,372,141 which is incorporated herein.

In accordance with the invention, utilization of BIA in a biological model for BCA (body composition analysis) provides an objective assessment of the study subject's (whole body or organ (regional)) volume and distribution of fluids and tissues, as well as the electrical health of the cells and membranes.

The characteristics of BIA include precision, accuracy, feasibility and economy. BIA may be applied to any area of interest, locally, regionally or to the whole body. It is non-offensive, causing no harm. It may be repeated freely, as desired, to illustrate change over time so that changes in physiology, progression of conditions, the response to disease and treatment intervention can be monitored and intervention modified or changed to improve the individual patient's response and outcome.

Some embodiments of the invention apply the IPG/BIA technology for assessment, prognosis, the burden of illness, of vitality of organs for transplant, vitality of organs from other species for human transplantation (xenotransplantation), and to monitor and assess the timing of death.

Organ vitality assessment is based upon the ability of a modified BIA for BCA to illustrate cellular architecture, the health of cells and their membranes by the measured resistance (R), reactance (X) and calculated phase angle (Pa).

Prior to harvest, regional/segmental measurements are used to detect and illustrate organ cellular integrity and the excursions of fluid volumes due to disease, response and treatment. Upon organ harvest, signal introduction electrodes are placed at/on/under the opposite lateral peripheral borders of the organ being assessed, and signal detection electrodes are placed at/on/under the superior and inferior borders of the organ being assessed for the first part of the initial measurement.

The values of R and X are measured, capacitance (C) and Pa calculated and recorded.

The signal introduction patient cable clips are then re-positioned or placed on the electrode superior and inferior borders of the organ being assessed, while the signal detection patient cable clips are re-positioned or placed on the electrode opposite lateral peripheral borders of the organ being assessed.

Further values of R and X are measured and Pa calculated and recorded. The values are then compared to normal values, and the organ is determined to be acceptable (vital) or not. If acceptable, prior to organ implant (transplantation or xenotransplantation), the sequence of the above steps is repeated with comparison being made to the electrical values which were measured and recorded upon organ harvest and after transplant engraftment to continue the evaluation of vitality and patient response. The values should be within an acceptable range of agreement denoting no further loss of organ vitality, and then the implantation is completed.

The same scenario is utilized for organs from different species.

For determination of the timing of death, whole body and/or regional measurements are made at predetermined intervals of time (preferably, but not necessarily, every other day) with R, X and Pa being measured, calculated and recorded. Frequency of measurement varies in proportion to the events being captured to include the progression of the underlying disease processes, the treatment interventions made and the normal changes of physiology. Initial values are compared to normal values and to those serially measured and recorded.

The uncorrectable loss of cell mass and membrane capacity, as evidenced by a reduction in X and Pa or by an uncorrectable and increasing disparity of ECW (extracellular water) volume being greater than ICW (intracellular water) volume and remaining uncorrectable, are the hallmarks of the progression to the death of the biological entity. Pa values consistently less than ˜4 degrees denote serious illness. Pa values consistently less than ˜2 degrees denote imminent demise.

One embodiment provides a method for determining illness of a biological entity, progression to death of said biological entity, and/or timing of death of said biological entity, comprising the steps of: taking whole body measurements of R, X, Pa, ECW and ICW at predetermined intervals of time; recording said whole body measurements; comparing initial values of said whole body measurements to normal values of said whole body measurements and to serially measured values of said whole body measurements; and determining from said comparison step hallmarks of said illness of said biological entity, said progression to said death of said biological entity, and/or said death of said biological entity.

Another embodiment provides a method of organ vitality assessment for transplantation of said organ being assessed, comprising the steps of: placing signal introduction electrodes at/on/under opposite lateral peripheral borders of said organ upon harvesting of said organ; placing signal detection electrodes at/on/under superior and inferior borders of said organ for a first part of an initial measurement upon said harvesting of said organ; measuring and recording first measured values of R and X and calculation of Pa of said organ in said initial measurement; then placing said signal introduction patient cable clips on the electrode at said superior and said inferior borders of said organ; placing said signal detection patient cable clips on said electrode at/on/under opposite lateral borders of said organ; measuring and recording second measured values of said R and X and calculation of Pa of said organ; and comparing said first and second values to normal values to determine if said organ is acceptable or not for said transplantation.

There will now be described one embodiment which provides a method and apparatus for use in detecting the presence and severity of illness, the effectiveness of treatment interventions, and the ability to change treatment to be more effective or aggressive; to optimize outcome, limit morbidity and mortality and illustrate the patient's prognosis.

The purpose of this embodiment is to empower the healthcare provider and the patient by detecting and characterizing the presence and nature of illness and injury to include episodic, serious, and non-episodic chronic illness and injury, its progression, and the effectiveness of treatment interventions and the prognosis of the patient.

There is provided a method and system for use in detecting the presence and severity of illness in diagnosing and treating a patient to optimize the treatment intervention and determine the prognosis of the patient. The system employs the use of Whole Body and/or Regional/Segmental Impedance Analysis to measure and calculate the patient's R, X and Pa and related electrical values at a healthy baseline, and thereafter in relation to the patient's complaints to evaluate the temporal or progressive nature of negative values or diminution of the measured values over time.

The system identifies the patient's healthy baseline measured electrical values and, during routine health examinations or when the patient complains of any symptoms or experiences any signs of illness or injury, illustrates excursion from the baseline values that may exceed a 30-day time frame or progressively diminish. Episodic illness and recoverable injury is characterized by a brief, less than 30 days, excursion below the baseline values and return to the baseline values. More severe illness, chronic disease and injury are characterized by progressive or rapid diminution of the measured values.

Once an effective treatment intervention is begun, the measured values will stabilize and then return to the baseline values indicative of the patient's positive prognosis. More effective treatment and a positive response are indicated by a more rapid return to baseline-measured values. If the values do not improve, a modified or more aggressive treatment intervention is indicated whose positive effectiveness will be indicated by the initial stabilization of the measured values and their subsequent return to baseline values. Prognosis is proportional to the speed and direction of the return of the measured value to or from the baseline values. A positive prognosis is indicated by a progressive and/or rapid return or continued to the measure baseline values. A negative prognosis is indicated by a progressive and/or rapid diminution of the measured values. The speed of loss or gain of the measured values is proportional to the return of health or the severity of the illness or injury. A neutral or stabilized measured value lower than the healthy baseline, over an extended period of time, greater than six months, indicates a new baseline, a less healthy condition and pre-disposition to future illness.

Frequency of measurements is in proportion to the severity of the process to be illustrated; more severe illness or injury, characterized by more severe symptoms, signs and negative laboratory findings and progressive and/or rapid diminution of the measured values, require more frequent measurements, daily and every other day. Less severe illnesses and injuries may be illustrated with weekly measurements.

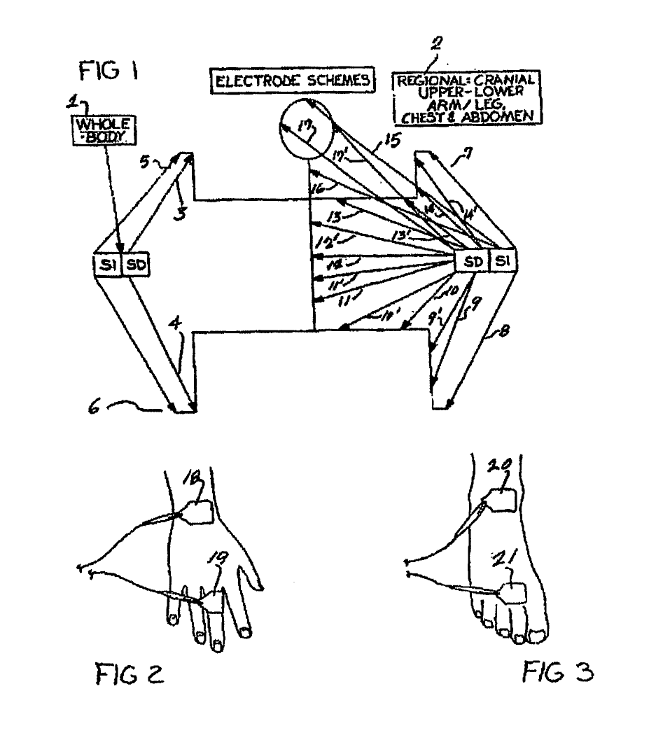

The first major inventive aspect will now be further explained with reference to FIGS. 1-3.

The primary study method for an IPG examination either Whole-Body 1 or Regional 2 is simple and straightforward. The patient/subject requires no advanced preparation for the study. However, the patient should not be diaphoretic, soaked in urine or any other surface liquid that would provide an alternative pathway for the conduction of the electrical signal that is the basis of the study.

The patient is counseled to lie quietly, motionless, and informed that the test will take less than five minutes if the patent is cooperative. The patient is generally placed in a supine position with arms and legs abducted about thirty degrees from the midline on a dry non-conductive surface. Whole Body 1 and Regional 2 studies require a tetrapolar electrode scheme in which placement of four (two pairs) surface, ECG electrodes in strict relation to anatomical landmarks at the wrist and ankle. If the patient's skin is either too dry or too oily, wiping the electrode placement area with an alcohol prep wipe is suggested. The right side of the body is generally used with the electrodes placed ipsilaterally. However if the patient's condition requires contra-lateral placement and alternative body positions, they can be utilized with the understanding and proviso that the same position will be repeated with all future measurements. The signal detection (SD) electrodes 3 or 4 must be placed with the greatest precision in relation to known anatomical landmarks on both the wrist and the ankle.

On the wrist, the superior linear border of the electrode, its top straight line, must equally bisect the ulnar stylus, bone prominence (bump) on the little finger side of the wrist with the tab of the electrode facing away from the body of the patient. The signal introduction (SI) electrodes 5 are placed distal from the SD electrodes 3 and must be kept at a minimum distance that equals or exceeds that of the diameter of the segment being measured (e.g., the wrist). This is most easily and efficiently accomplished by using the distal phalanx of the middle finger, just proximal to the nail.

On the ankle, the SD electrode 4 is placed so that the superior linear border equally bisects the medial malleolous (the bump on the big toe side of the ankle) with the tab facing outwards from the patient. Care should be exercised to use the medical malleolous because the lateral malleolous (the bump on the little toe side of the ankle) is inferior or below the medial malleolous landmark. The SI electrode 6 is placed on the big toe, as shown in FIG. 1.

The IPG is connected via patient cable leads with strict attention paid to SI and SD leads connected to SI and SD electrodes. The device is energized and the values of R and X are measured individually, allowing a moment (10-15 seconds) to settle, and then are recorded. The electrodes are carefully removed so as not to injure friable skin or contaminate the examiner.

The IPG may use a 500-800 micro-amp constant current electrical source at 50-kilohertz frequency. A RJL Systems, Inc. manufactured instrument system may be used for both Whole Body 1 and Regional 2 measurements, but variable currents, frequencies, electrode arrays and instrument may also be used.

For Regional 2 measurements, the patient is prepared in the same manner as with a Whole-Body 1 examination. For in-vivo Regional 2 measurements of the chest, abdomen or extremities (arms/legs, left-right, upper or lower), the SD electrodes 7 are placed superiorly and inferiorly in precise relation to the area of interest. The distance between the SD electrodes is precisely measured and recorded in centimeters. The skin is marked with a surgical pen to assure accurate and reproducible electrode placement for serial measurements. The SI electrodes 1 are best placed in the standard Whole-Body locations. This requires a specialized patient cable with adequate distance or throw, about 18″ of length allowed, between the insertion point into the patient cable to and from the clip ends. The IPG is connected via the patient cables with strict adherence to the SD lead to the SD electrode and the SI lead to the SI electrode. The measured values are recorded and the electrodes carefully removed.

The measured values, R, X and Pa (calculated), are recorded, archived and graphically presented, compared to normal values and then followed serially to illustrate change over time and illuminate the processes of disease progression and response to treatment. The frequency of serial measurements is proportional to the dynamic of the event to be captured. If at all possible, a baseline study value is particularly desirable.

Disorders characterized by dynamic shifts of extracellular fluid volumes require more frequent measurements, often prior to and after a procedure or treatment such as a patient requiring hemodialysis, aggressive diuresis in organ failure or repletion of fluids in acute dehydration or trauma. The measured R is inversely proportional to the extracellular fluid volume of the patient. When R decreases; fluid volume has increased. When R increases, fluid volume has decreased. Once an initial R value is established by baseline or first study, subsequent measurements illustrate the patient's course and response to disease progression and the effectiveness of the selected treatment intervention. The severity of the disease or insult condition evidenced by the speed of the excursion from baseline or initial measurement value. Fluid changes that move more than 50 ohms in a 24-hour period are severe and indicate a more acute and serious condition than those that move 50 ohms in a week's time indicative of a more chronic condition. Both conditions require intervention. Chronic insidious changes are as adverse to survival as more rapid changes. These changes may be evidenced in both Whole Body 1 and Regional 2 measurements. Whole Body 1 measurements are more general in their value, indicative of conditions and events that encompass the organism as a whole, such as cardiac or renal failure and acute dehydration. Regional 2 measurements provide a site-specific assessment of fluid volumes, such as those found with pleural effusion in the chest, ascites in the abdomen or even cerebral edema. The changes of measured electrical values precede changes seen on x-ray, physical examination, or from laboratory studies.

Thoracic R values that are increasing indicate a drying chest. Decreasing R values indicate additional accumulation of fluid. These changes indicate the improvement or worsening of disease conditions and the individual's response to treatment and its effectiveness. The extent and aggressiveness of therapy can be altered and modified to “optimize” the beneficial effects.

X values are proportional to the number and integrity (health) of cell mass and corresponding cell wall membranes so when cells increase or decrease, X values follow. The cells that change in this manner are those of the somatic and visceral protein tissues, such as skeletal musculature organs such as the liver, spleen, lungs, heart stomach and intestines. Cellular alterations are generally slower to occur and are affected by metabolic and specific disease processes (inflammation, infection, rejection and/or chemical imbalances, trauma, insult and/or injury). Overly aggressive diuresis, excessive hemodialysis or cellular targeted pathologies such as Rhabdomyolysis can all result in rapid, days versus a week, changes in cell mass, membrane status and measured X. Excursions from the baseline or initial measurement value indicate the type and progression of disease and/or the effectiveness of treatment interventions. Increased cells (membranes) and anabolic metabolism are evidenced by a rise in X, generally a sign of improvement. A slowly decreasing X indicates a negative or catabolic metabolism condition. A more precipitous and rapid decrease in X is indicative of unique conditions that rapidly affect cells and their membranes, such as the effect of Rhabdomyolysis skeletal muscle or rejection or infection of an organ system.

Regional measurement values of X are used for these disease specific investigations while whole body values are used for the assessment of metabolic evaluation.

A derivative of the measured values of R to X is the arc tangent of X to R expressed in degrees or Pa. Pa is the cumulative expression of the changes and ratios of cell mass and extracellular fluid that result from disease, insult and/or treatment intervention and can by itself be used to gauge the severity and progression of pathologies and the effectiveness and benefits of treatment. Pa reflects the condition of the cell membrane and its mediation between the intra and extracellular milieus. A positive prognosis or more healthy and vital organ is indicated by an increasing Pa. A poor prognosis or less vital or healthy organ is associated with a Pa decrease. Pa has been correlated with survival and the timing of non-acute death. Pa can be derived from both whole body and regional measurements and followed serially to establish prognosis.

Treatment interventions can be measured for their effectiveness on the individual patient by following Pa. More effective treatments are evidenced by an increasing Pa, while those less effective are seen as producing little or no increase. Once Pa persistently degrades to and stays below 4 degrees, the patient is seriously ill and treatment should be aggressive and modified to be effective and optimal. If Pa does not stabilize or increase through multiple iterations of treatment, a curative or restorative treatment goal outcome is doubtful. A Pa of persistently less than 2 degrees is associated with pending and unavoidable mortality and a need for discontinuation of curative or restorative treatment effort, and for the initiation of palliative treatment, care and comfort. Admission to a hospice can be objectively based upon Pa monitoring providing the patient with improved end-of-life care and comfort.

FIG. 2 illustrates how electrodes may be placed on the hand for the BIA Testing Procedure. The signal electrode edge 8 is placed on an imaginary line bisecting the ulna head (bone on little finger side of wrist). The SD electrode 9 is placed on the first joint of the middle finger.

FIG. 3 illustrates how electrodes may be placed on the foot. The SD electrode edge 10 is placed on an imaginary line bisecting the medial mellealus (bone on big toe side of ankle). The SD electrode 11 is placed on the base of the second toe.

The exam area should be comfortable and free of drafts. The exam table surface must be non-conductive and large enough for the subject to line supine with the arms 30 degrees from the body, and legs not in contact with each other.

The subject should not have exercised or taken a sauna within 3 hours of the study. The subject's height and weight should be accurately measured and recorded. The subject should lie quietly during the entire test. The subject should not be diaphoretic or wet from sweat or urine.

The subject should not have a fever or be in shock or if such is present comparison to serial measurements should be made only to those made in the same or similar conditions. The study and testing procedure should be explained to the subject.

The subject should remove the shoe and sock and any jewelry on the electrode side (generally the study is completed on the right side of the body). The body side (left or right) should always be used subsequently.

The subject should lie supine with the arms 30 degrees from the body with legs not touching.

The electrode sites may be cleaned with alcohol, particularly if the skin is dry or covered with lotion. The electrodes and patient cables are attached as shown in FIGS. 2 and 3. The analyzer is turned on, making sure the subject refrains from moving. When the measurements have stabilized, record the displayed R and X with the subject's name, age, gender, height and weight. The entire testing time is less than 5 minutes—the BIA analyzer is on for less than one minute. The results are available immediately from the software program. The study may be repeated as often as necessary.

The invention also embraces the features of using the invention for various areas of interest, for example, whole-body, thoracic, abdominal, extremity, etc.

IPG diagnostics (IPGDx™) are based upon the illustration of “cellular” level physiology through their measured electrical equivalents. The subject becomes the only unknown part of an electrical circuit.

Based upon the purpose of the study the patient's whole-body or a regional section will be studied. A 4-electrode tetrapolar scheme of 2 pairs of surface ECG-type stick-on electrodes is placed in relation to prominent and/or carefully noted anatomical landmarks.

One pair introduces the electrical field; the SI electrodes. The second pair detect the changes in the electrical field that result from the patient being part of the circuit and are placed in relation to the area of interest either whole-body or regional.

A patient cable is connected to the electrodes when necessary the patient cables are moved from SI electrodes to SD electrodes to make the second measurement of a regional measurement or in-vitro organ assessment and to the plethysmograph. The plethysmograph has two purposes; to generate a constant precise electrical signal; and to measure the ‘patient segment’ of the circuit.

The electrical signal may beat a fixed or variable frequency. The voltage is generally fixed at ˜500 to 800 micro-amps. Both are adjusted to meet the specific requirements of the physiologic event to be captured.

The frequency is maintained above the threshold that would stimulate, disturb or insult the tissues of the subject. The signal strength is maintained at a constant value to accommodate subjects of various physiognomies.

The measured values of R and X are measured and recorded along with patient identification, age, gender, height, weight and if a regional measurement is performed the distance between the SD electrodes and the area of interest is identified.

The distance between the SD electrodes is important as the area of interest must be between the detection electrodes and they must be configured accordingly to provide the depth of measurement appropriate to the phenomenon sought or captured. A peripheral event in the skin, such as capillary perfusion, is seen with the SD electrodes close to each other. The study of an internal structure requires the distance between the electrodes to be increased to address its anatomical location.

For instance in studying the liver, two pairs of SD electrodes would be used to that would approximate the superior/inferior borders and the lateral/medial borders to record measured values from the entire organ. The SI electrodes must be at least the distance from the detection electrodes that is greater than the diameter of the segment of the body to which they are applied.

They are best kept on the hand and foot, but may be applied superiorly and inferiorly to the area of interest as long as they are at a distance greater than the diameter of the body segment. This is due to the need for the electrical field to be fully and adequately distributed through the area of interest to complete the circuit and include the area of interest within the detection electrode array.

The measured R and X are a series circuit model, and are transformed mathematically to the equivalent parallel circuit model of the body. The values of R, X and Pa correspond to physiologic variables of biology. The R value is inversely proportionate to extracellular water. The X value is proportional to cell mass, as the plasma bi-lipid membrane acts as a capacitor and reflects the intracellular water volume and body cell mass (combined somatic and visceral proteins). A single measurement is essentially a ‘snap-shot’ in time of the conditions encountered.

The measured values may be compared to ‘normal’ and assessment of excess, equality or absence can be made. Through serial assessments change over time can be documented.

The technique is highly reproducible as it is a simple electrical circuit, which does not change and is well understood, while the subject part of the circuit is constantly changing, so the changes in the measured values are inherent to those of the subject.

A small error is possible with misplacement of the SD electrode pair by the examiner. Prominent anatomical landmarks, measured values and simply marking the skin can be used to minimize this effect. This operator error is ˜2% or less and is managed through training, testing and specialized electrode arrays.

The technique is best suited to illustrate change over time as the condition of interest may change; such as disease progression or the response to treatment interventions. In this manner the results become guides to assessing the effectiveness of treatment, the effects that changes in the treatment intervention may induce and the patients overall response. The particular value of the results is that they are cellular level values.

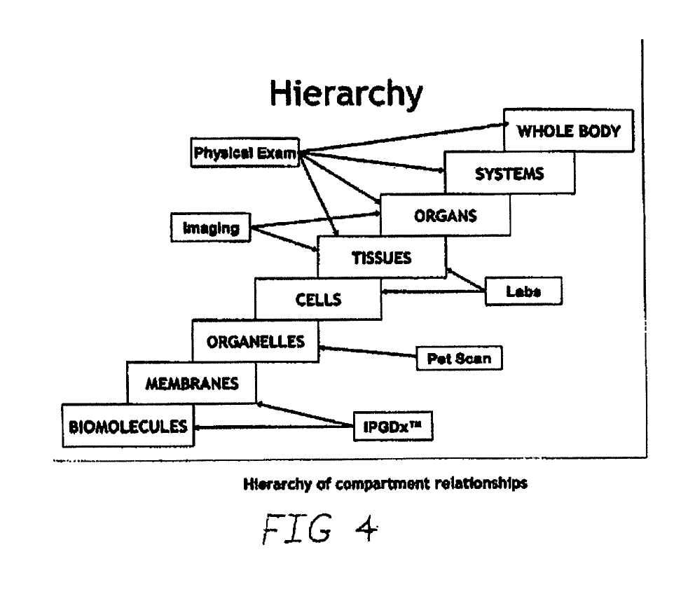

With reference to FIG. 4, the body is organized in an ensemble of compartments and this hierarchy of organized functionally and spatially distinct compartments range from the microscopic (intracellular) to macroscopic levels (gross whole body). The transport process and communication between each level is mediated through cell membranes. On a microscopic level, physiologic interactions are mediated through channels, carriers and pumps; on the macroscopic level, by skeletal musculature (somatic body cell mass). Pathophysiology from any etiology; insult, injury or disease process is evidenced on the membrane transport system gone awry.

The data resulting from the impedance (Z) measurement is more sensitive, specific and valuable than traditional indices because it is the pre-cursor to these ‘down-stream’ occurrences. This membrane level dataset provides an invaluable bridge seemingly prescient as changes at this level of the hierarchy occur to those downstream.

Prior to a change in a blood chemistry value, the development of inflammation, infection, rejection or the prominence of a physical sign, finding on an imaging study or patient complaint of a symptom a membrane transport process is askew. This change can be noted through the impedance study and correlated with the more gross and later developing findings and be used to provide better interventions sooner.

IPGDx™ test results provide information about:

Fluid volumes and shifts between the intra and extracellular milieu

Nutrition status

Cell membrane health

Metabolism

Infection

Inflammation

The cellular architecture of
Organs
Muscles
   These data are able to be used to evaluate;

Presence of disease
Systemic
Regionally

Progression of disease

Response to pharmacologic treatment intervention

Need to change or terminate treatment

Patient's prognosis

Organ vitality and function
In vivo
   Hepato-cellular architecture
      Cirrhosis
      Fibrosis
      Steatosis
   Lung water (Pulmonary edema)
In vitro
   Organs for transplant
      Cellular architecture

Timing of non-acute death

Outcome

Classification of potential treatment outcome
Curative
Restorative
Palliative

The invention covers not only in vitro transplantation applications, but also impedance In vivo assessment of organ vitality, e.g., liver (kidney, lung).

With the patient in a dorsal recumbent position; lying on their back on a non-conductive surface; Standard whole-body measurement is made with signal introduction electrodes placed on the distal Right Hand and Foot, SD electrodes placed in relation to ulnar stylus at wrist and medial malleolous in ankle; measurement of R and X taken and recorded

SD electrodes are placed in relation to superior/inferior borders of liver (kidney or lung) and lateral/medial borders of liver measurement of R and X taken and recorded from each set.

The measured values are converted to their equivalent parallel circuit model and phase angle is calculated, they are compared to “normal” values and previously measured values if available over time as they change in response to treatment and disease progression.

The presence of pathophysiology such as; cirrhosis, fibrosis and/or steatosis or ascites is evidenced by the measured values. As opposed to liver biopsy the impedance assessment is noninvasive, samples the entire organ (versus 1/5,000^{th}) and is without complication (versus a rate of 0.59%).

For the second major aspect of the invention, the following terminology applies.

The term ‘live’ foodstuffs means any and all living organisms including meats, fish, fowl, fruits and vegetables.

The term ‘biological entity’ means any and all portions, carcass, parts or whole of a live or previously-live organism.

The term ‘subject’ means that portion, segment, ‘cut’ or whole biological entity studied.

The term ‘electrode scheme’ means any and all configurations utilized to introduce and measure the electrical signal and corresponding voltage drop by placement on the subject's surface, around said surface, into said subject and/or through placing said subject onto the electrode configuration singularly or as part of another appliance.

The term ‘average’ means the product of the statistically valid sample size number divided into the measured values.

The term ‘normal’ means the product of the average peculiar to and comprised of but not limited to a defined group; age, gender, species, or cut.

The term ‘optimal’ means the best or most favorable value; which may be obtained subjectively individually or collectively or it may be obtained objectively as compared to a ‘criterion’ or ‘gold-standard’ designated and agreed upon by professional, experts and those ‘experienced’ in the field of endeavor and by personal selection of a value on that objective scale an individual may express and select their personal optimal value.

The term ‘individual’ means those findings peculiar to a single subject or to a uniformly collective group of individual subject's assigned to a group based upon a preponderance of similar and agreed upon characteristics such as but not limited to; genus, species, cut, breed, or other such recognized characters of physicality and composition.

The term ‘meat’ means bovine (Bos), porcine, lamb (Ovis Aries), buffalo, bison camel, goat (Capra Hircus) equine, donkey, llama, reindeer and yak.

The terms ‘fowl’ or ‘poultry’ means chicken, turkey, duck, geese, guinea fowl and swan.

The term ‘external appliance’ includes but not limited to scales, refrigerators, display, and/or packaging materials, methods, device or systems and portable temperature controlled appliances, and cooking appliances.

The term ‘freshness’ is a dynamic characteristic of vitality progressively decreasing after death with processing through proteolysis, decomposition which may be slowed and/or controlled by preservation through chemical, temperature, mechanical, humidity, air flow control, and light exposure restriction.

The term ‘Palatability Index’ (Palatability: tenderness, flavor and juiciness) are the objective results scaled to the characteristics of the foodstuff and reported in priority of importance; safe versus unsafe and then as varying degrees of palatability and used to support subjective decisions of producers, purveyors, merchants, preparers, and consumers of the foodstuff for the purposes of preference, pricing, acquisition, safety, health, determination of fresh or frozen, or selection for culinary preparation.

The invention provides a method and system to obtain and use the measured values and products of BIA as an objective means to equivalently illustrate electrically, various physiological characteristics, and upon which characterization the palatability of foodstuffs can be objectively and subjectively described and compared and practically utilized.

The method of BIA measurement may comprise various configurations so as to accommodate the diversity of foodstuffs so measured to the extent that the interface with the foodstuff (electrode array/scheme, electrical power management (frequencies, current and voltages)) and circuit models (series and/or parallel) may be varied as such to incorporate the subject foodstuff within the controlled electrical circuit or field of the BIA measurement comprised in such manner as to complete the measurement.

The interfaces for electrode array/scheme may be comprised of; placement of the studied foodstuff within a generated electrical field array, on an electrode scheme array, placing the electrode array about around or as comprised in such configuration as to measure ‘capture’, characterize and illustrate the unique geometry and traits of the subject foodstuff in its entirety or as possible the electrode scheme and array may be introduced directly into the study subject foodstuff, and/or that such electrical power management configurations may be comprised of fixed or variable frequencies, currents and voltages and circuit models (series and/or parallel) and that the measured and calculated values be comprised of such values and sampling rates to adequately capture, characterize and illustrate the unique geometry and traits of the subject foodstuff in its entirety.

The electrical signals used to measure and calculate the Z, R, X, capacitance (C) and Pa may comprise multiple schemes based upon the type and geometry of the foodstuff; a mono or singular frequency, multiple frequencies, or a spectroscopic illustration across a segment or band of frequencies.

The measured and calculated electrical values comprised of Z, R, X, C and Pa are related to the comprised physiological values of fluid; volume and distribution, the cell mass; volume, character and membrane vitality as related to the unique and inherent characteristics palatability (flavor, juiciness and tenderness) of the studied subject foodstuff and reported in such a manner as to provide a basis for objective assessments and subjective interpretation of said comprised values for foodstuff product; safety grading, pricing, handling, management and disposition.

The invention provides a method and system for the use of BIA in the electrical measurement of a biological equivalent model of ‘live’ foodstuffs or ‘biological entities’ to provide an objective assessment and scale of palatability to include safety, freshness, juiciness, flavor and tenderness as related to the characteristics, volume and distribution of fluids, tissues and cells as well as the electrical vitality of cells and cell membranes through the measurement of Z, R, X and C and the calculation of Pa at a fixed or variable electrical frequency, current and voltage through a tetrapolar electrode scheme placed on, around and/or in or with the subject placed upon the array or by placing the study subject within a electrical field or a portion thereof by placing said foodstuff biological entity or a portion thereof onto an electrode configuration singularly or as comprised as part of an external appliance; such as part of a scale; refrigerator or a portable temperature controlling device, packaging or display, the study subject as measured individually; compared to normal, average and optimal values and as tracked serially over time and compared to changes from the initial measurement.

The invention also provides a method and system for determining the palatability of a portion or whole live or previously live foodstuff such as a meat, fish, fowl, fruit or vegetable, to grade its characteristics (palatability), quality and salability, and to support decisions regarding its disposition, preparation and presentation and cost and consumption.

The invention can use a modification of a BCA to include, but not limited to, impedance measuring instrumentation capable of measuring Z, R and X for the calculation of C and Pa from selected singular or mono-frequency, multiple frequencies and/or impedance spectroscopic analysis or changes in current, power and voltage.

With the invention, utilization of BIA in a biological model provides an objective assessment of the study subject's (whole or section of the biological entity) volume and distribution of fluids, tissues and cells, as well as the electrical health and vitality of the cells and membranes.

The characteristics of BIA include precision, accuracy, feasibility and economy. BIA may be applied to any subject whole or an area of representative sample or interest to be studied and examined for palatability; the carcass during processing, a section thereof, regionally, or to the whole biological entity. It is non-offensive, causing no harm. It may be repeated freely, as desired to capture various dynamic changes unique to the variety of live foodstuffs (biological entities), to illustrate initial values and change over time so that progression of conditions can be monitored and changes that effect palatability determined during transport, preservation, packaging and transfer. The specific value of BIA is in its precision of measurement and the significance of the electrically measured products illustration of the biological foodstuff entities equivalent physiological variables of fluid, tissue and cells volume and distribution, cell membrane volume and vitality, derivative values initially and comparison to average, optimal, normal, and subsequent individual values and changes serially over time.

Based upon the individual genus, type; species, ‘cut’ or sample of the biological foodstuff entity, palatability is determined by the baseline values, and changes thereto (rate, zenith and nadir) of the measured and calculated values initially and over time. The properties of the electrical values directly relate to biological equivalents. R is inverse to water content (juiciness) so an increasing R value is indicative of water loss. A decreasing R value is indicative of water accumulation. X is proportional to cell mass. A decreased X is indicative of cell membrane loss through such processes (naturally occurring or artificially induced) as fragmentation or proteolysis; a diminution of X and/or a change in the rate of the diminution from a zenith towards a nadir is indicative of optimal palatability (tenderness, flavor and juiciness) which may progress beyond that nadir of palatability and become non-palatable. Comparison of the X of one sample of the same genus and species, section and cut of a biological entity to another sample of the same genus and species, section and cut of a biological entity illustrates a comparative scale of palatability. A consumer may have a subjective selection of a particular palatability scale value which translates to his/her individual desire and preference.

The invention also provides a method of palatability assessment of a foodstuff biological entity being assessed, comprising the steps of: placing SI and SD electrodes on/in or/around the foodstuff subject studied such as, on or within the opposite lateral peripheral borders of the organ upon selecting or harvesting of the biological entity; placing SI and SD electrodes on/in or/around or within the superior and inferior borders of the biological entity for a first part of an initial measurement upon the selection and harvesting of the biological entity; measuring and recording the first values of Z, R and X and calculating C and Pa of the biological entity in the initial measurement; then placing said SI and SD electrodes on/in or/around or within the superior and the inferior borders of the biological entity; placing the SI and SD electrodes on/in or/around or within the opposite lateral borders of the biological entity; measuring and recording second values of Z, R and X and calculating C and Pa of the of the biological entity; and comparing the first and/or second values to normal, average, optimal and individual values to determine the scale of palatability of the biological entity and by serial measurements if palatability has changed in response to time (aging or preservation), external intervention (chemical, electrical or mechanical) or not for and then serially additional series of the measurements and calculations are repeated at predetermined intervals based upon the individual characteristics of the biological entity, the time it was harvested and the manner it is stored and transported.

Alternative electrode scheme arrays include alternative external placements to include: circumferential wrapping, multiple placement locations and placement of the study subject on any such array.

Another alternative is the internal placement of an electrode array in which the electrodes are introduced into the study subject at various locations, depths and configurations.

Another variation in measurement is the entry or placement of the study subject within an electrical field (such as generated within a solenoid) and through a fixed or scanning process measures the electrical properties as related to the water and cell content as they relate to palatability.

One embodiment is the assessment and illustration of the preservation or aging process to provide objective and subjective scaling to price, sell and market based on results.

Another embodiment is to grade and report such palatability values for the purpose of pricing and salability in a grocery.

Another embodiment is a sales and marketing tool by presenting palatability as a menu/product variable available from a merchant, such as a meat producer, grocer or restaurateur.

Another embodiment is utilization by the consumer at home, point of purchase or point in time of preparation or consumption in the assessment of palatability of foodstuffs.

Another embodiment forms part of an external appliance, such as a scale, refrigerator, display or packaging system or portable temperature-controlled appliance to determine the effectiveness of preservation.

Another embodiment is the determination when the foodstuff is not palatable, safe or unsafe.

A specific purpose of the invention is in its application to the following example; a sub-primal loin cut section is removed two days after harvest (post-mortem) from a USDA Premium Choice beef carcass during in-plant fabrication.

The tenderloin sub-primal while hanging has four stainless steel electrode quality skewer probes placed through it, the first and outer pair at the beginning (top) and end (bottom) of the loin, becoming the BIA signal introduction electrodes and within that first pair a second pair is placed to the approximated beginning and end of the ‘strip loin’ longissimus dorsi becoming the BIA signal detection electrodes The IPG is connected to the electrodes, energized and the readings of R and X are taken, automatically entered identified, date and time-stamped into the instrument. The IPG is disengaged and the electrodes probes removed and calculations of Z, C and Pa are made and converted into a corresponding value of a palatability index for that specific cut of beef (in this instance a 4.5 on an acceptable range of from 3 to 6) and reported.

Throughout the aging or preservation process selected for this cut the measurement procedure is repeated every 4 days for 16 days (four measurements that can coincide with the transit of the meat from processor, to purveyor to merchant provider; retail grocer or restaurateur) and the newly determined values are compared to the initial values to establish the rate of change and the rate of continued testing, every other day or every day based on progression towards the optimal value range for this cut at which time the meat is available for final sale, disposition, processing and preparation and consumption as a end-user consumer may select their individual subjective preference value from the determined palatability index (in this instance a final index value of 9, with a premium tenderness range of from 7 to 10).

Although the invention has been described in detail in the foregoing only for the purpose of illustration, it is to be understood that such detail is solely for that purpose and that variations can be made therein by those of ordinary skill in the art without departing from the spirit and scope of the invention as defined by the following claims, including all equivalents thereof.

1. A method of determining and monitoring palatability and freshness of a foodstuff biological entity including at least a portion of a live or previously-live organism, comprising the steps of:
subjecting said foodstuff biological entity to bioelectrical impedance analysis for measurement and composition analysis; and
utilizing results of said subjecting step to illustrate an objective scale of palatability and freshness of said foodstuff biological entity.

2. A method according to claim 1, wherein:
said bioelectrical impedance analysis includes measurement and/or calculation of resistance, reactance, impedance, capacitance, and/or phase angle of said foodstuff biological entity.

3. A method according to claim 1, wherein:
said utilizing step also determines a value of an “Electrical Freshness”, “Palatability Index” or “EFRESH” certification for said foodstuff biological entity.

4. A method according to claim 2, including:
placing said foodstuff biological entity or a portion thereof in an electrical field; and
taking said measurements through a fixed or scanning process.

5. A method according to claim 1, wherein:
said bioelectrical impedance analysis includes measurement and/or calculation of resistance, reactance, impedance, capacitance, and/or phase angle of said foodstuff biological entity as determined through measurement by mono or multiple frequencies or spectroscopic analysis and by series and/or parallel circuit models; and
using voltage and current sufficient to accommodate the geometry of said foodstuff biological entity.

6. A method according to claim 1, wherein:
said method is utilized to determine optimal aging, curing, and/or processing of said entity; and
said subjecting step includes subjecting said entity to bioelectrical impedance analysis for measurement, composition analysis, and serial tracking and grading of aging or preservation of said entity and determination of aging or preservation (intentional or incidental) beyond palatability.

7. A method according to claim 3, wherein:
said method is utilized to determine optimal aging, curing, and/or processing of said entity; and
said subjecting step includes subjecting said entity to bioelectrical impedance analysis for measurement, composition analysis, and serial tracking and grading of aging or preservation of said entity and determination of aging or preservation (intentional or incidental) beyond palatability.

8. A method according to claim 6, including:
placing said foodstuff biological entity or a portion thereof in an electrical field; and
taking said measurements through a fixed or scanning process.

9. A method according to claim 6, wherein:
said bioelectrical impedance analysis includes measurement and/or calculation of resistance, reactance, impedance, capacitance, and/or phase angle of said foodstuff biological entity; and
including the step of comparing said measurements and calculations to normal values, average values, optimal values, and/or individual values, and in response to time and/or external influences purposeful or incidental.

10. A method according to claim 7, wherein:
said bioelectrical impedance analysis includes measurement and/or calculation of resistance, reactance, impedance, capacitance, and/or phase angle of said foodstuff biological entity; and
including the step of comparing said measurements and calculations to normal values, average values, optimal values, and/or individual values, and in response to time and/or external influences purposeful or incidental.

11. A method according to claim 7, including:
placing said foodstuff biological entity or a portion thereof in an electrical field; and
taking said measurements through a fixed or scanning process.

12. A method according to claim 6, wherein:
said bioelectrical impedance analysis includes measurement and/or calculation of resistance, reactance, impedance, capacitance, and/or phase angle of said entity as determined through measurement by mono or multiple frequencies or spectroscopic analysis; and
including the step of comparing said measurements and calculations to normal values, average values, optimal values, and/or individual values, and in response to time and/or external influences purposeful or incidental.

13. A method for determining palatability and freshness of a foodstuff biological entity, changes of palatability of said biological entity, and/or timing of optimal palatability, loss of the palatability of said biological entity and/or illustrating an objective scale of palatability from which a producer, purveyor, merchant, preparer or consumer may objectively and subjectively apply individual tastes and select from said scale their preference, comprising the steps of:
providing normal, average, optimal and individual measured values of resistance, reactance, capacitance and phase angle, of a sample subject studied of said foodstuff biological entity;
measuring initial values of impedance, resistance, reactance, capacitance and phase angle of said sample subject foodstuff biological entity;
taking measurements of impedance, resistance, reactance, capacitance and phase angle, at predetermined intervals of time based upon said sample subject foodstuff biological entity;
recording said measurements;
comparing initial values of said measurements to normal values of said measurements and to serially measured values of said measurements; and
determining, from said comparison steps, hallmarks of palatability of said foodstuff biological entity, said progression of changes in palatability and freshness of said biological entity, to a specific individual Palatability Index or Electrical Freshness value which may be reported and found as the inherent average, normal, optimal and/or safe individual characteristics of said foodstuff biological entity or portion thereof.

14. A method according to claim 13, including:
measuring initial values of impedance, resistance, reactance, capacitance and phase angle of said sample subject biological entity as determined by mono or multiple frequencies or spectroscopic analysis and/or at various current, voltage and power, and by series and/or parallel circuit models requirements as to accommodate the inherent characteristics of the subject foodstuff biological entity.

15. A method according to claim 13, including:
placing said foodstuff biological entity or a portion thereof in an electrical field; and
taking said measurements through a fixed or scanning process.

16. A method according to claim 13, including the steps of
determining a first value of a “Palatability/Freshness Index” from said measured initial values of impedance, resistance, reactance, capacitance and phase angle of said sample subject foodstuff biological entity;
determining a second value of said “Palatability/Freshness Index” from said measurements at said predetermined intervals of time; and
determining third values of said “Palatability/Freshness Index” based upon said comparison steps.

17-20. (canceled)

21. A method of organ and tissue vitality assessment in a biological entity, human, animal, fruit or vegetable, comprising the steps of:
utilizing bioelectric impedance analysis in a biological model for composition analysis; and
using the results of said utilizing step to provide an objective assessment of volume and distribution of fluid and tissues, as well as electrical health of cells and membranes of said organ or tissue of any biological entity.

22. The method according to claim 21, including the step of:
utilizing a modified bioelectric impedance analysis for composition analysis to assess the health of cells of said organs and tissues or the biological entity by the measured reactance thereof.

23. A method according to claim 21, wherein upon harvesting, processing, preserving, treating or transporting said organ or tissue or meat, fish, fruit or vegetable from the donor or source, including the steps of:
placing signal introduction electrodes on opposite lateral peripheral borders of said organ tissue, meat, fish, fruit or vegetable;
placing signal detection electrodes at superior and inferior borders of said organ tissue, meat, fish, fruit or vegetable for a first part of an initial measurement;
measuring and recording first values of resistance and reactance and calculating the phase angle of said organ tissue, meat, fish, fruit or vegetable in said initial measurement;
then placing said signal introduction electrodes on said superior and said inferior borders of said organ tissue, meat, fish, fruit or vegetable;
placing said signal detection electrodes on said opposite lateral borders of said organ tissue, meat, fish, fruit or vegetable;
measuring and recording second values of said resistance and said reactance and calculating the phase angle of said organ tissue, meat, fish, fruit or vegetable; and
comparing said first and second values to normal values to assess vitality.

24. A method according to claim 22, wherein upon arrival of said organ tissue, meat, fish, fruit or vegetable at the location of the recipient including the steps of:
placing signal introduction electrodes on opposite lateral peripheral borders of said organ, tissue, meat, fish, fruit or vegetable;
placing signal detection electrodes at superior and inferior borders of said organ, tissue, meat, fish, fruit or vegetable for a first part of an initial measurement of said organ, tissue, meat, fish, fruit or vegetable;
measuring and recording first values of resistance and reactance and calculating the phase angle of said organ, tissue, meat, fish, fruit or vegetable in said initial measurement;
then placing said signal introduction electrodes on said superior and said inferior borders of said organ, tissue, meat, fish, fruit or vegetable;
placing said signal detection electrodes on said opposite lateral borders of said organ, tissue, meat, fish, fruit or vegetable;
measuring and recording second values of said resistance and said reactance and calculating the phase angle of said organ, tissue, meat, fish, fruit or vegetable; and
comparing said first and second values to normal values to assess vitality of said organ, tissue, meat, fish, fruit or vegetable.

25. A method according to claim 23, wherein prior to preservation, sale, implantation, treatment and/or consumption of said organ tissue, meat, fish, fruit or vegetable into the recipient including the following additional steps:
again placing said signal introduction electrodes on said opposite lateral peripheral borders of said organ tissue, meat, fish, fruit or vegetable;
again placing said signal detection electrodes at said superior and said inferior borders of said organ tissue, meat, fish, fruit or vegetable;
measuring and recording third values of resistance and reactance and calculating the phase angle of said organ tissue, meat, fish, fruit or vegetable;
then again placing said signal introduction electrodes on said superior and said inferior borders of said organ tissue, meat, fish, fruit or vegetable;
again placing said signal detection electrodes on said opposite lateral borders of said organ tissue, meat, fish, fruit or vegetable;
measuring and recording fourth values of said resistance and said reactance of said organ tissue, meat, fish, fruit or vegetable; and
comparing said first and second values to said third and fourth values to determine if the values are within a predetermined acceptable range of agreement denoting no further loss of said organ tissue, meat, fish, fruit or vegetable vitality.

26. A method according to claim 24, including the following additional steps:
again placing said signal introduction electrodes on said opposite lateral peripheral borders of said organ tissue, meat, fish, fruit or vegetable;
again placing said signal detection electrodes at said superior and said inferior borders of said organ tissue, meat, fish, fruit or vegetable;
measuring and recording third values of resistance and reactance and calculating the phase angle of said organ tissue, meat, fish, fruit or vegetable;
then again placing said signal introduction electrodes on said superior and said inferior borders of said organ tissue, meat, fish, fruit or vegetable;
again placing said signal detection electrodes on said opposite lateral borders of said organ tissue, meat, fish, fruit or vegetable;
measuring and recording fourth values of said resistance and said reactance and calculating the phase angle of said organ tissue, meat, fish, fruit or vegetable; and
comparing said first and second values to said third and fourth values to determine if the values are within a predetermined acceptable range of agreement denoting no further loss of said organ tissue, meat, fish, fruit or vegetable vitality.

27. A method of organ, tissue, meat, fish, fruit or vegetable vitality assessment, comprising the steps of:
placing signal introduction electrodes on opposite lateral peripheral borders of said organ, tissue, meat, fish, fruit or vegetable;
placing signal detection electrodes at superior and inferior borders of said organ, tissue, meat, fish, fruit or vegetable for a first part of an initial measurement of said organ tissue, meat, fish, fruit or vegetable;
measuring and recording first values of resistance and reactance of said organ, tissue, meat, fish, fruit or vegetable in said initial measurement;
then placing said signal introduction electrodes on said superior and said inferior borders of said organ tissue, meat, fish, fruit or vegetable;
placing said signal detection electrodes on said opposite lateral borders of said organ, tissue, meat, fish, fruit or vegetable;
measuring and recording second values of said resistance and said reactance of said organ, tissue, meat, fish, fruit or vegetable; and
comparing said first and second values to normal values to assess vitality of said organ, tissue, meat, fish, fruit or vegetable.

28. A method according to claim 27, including the following additional steps:
again placing said signal introduction electrodes on said opposite lateral peripheral borders of said organ, tissue, meat, fish, fruit or vegetable;
again placing said signal detection electrodes at said superior and said inferior borders of said organ, tissue, meat, fish, fruit or vegetable;
measuring and recording third values of resistance and reactance and calculating the phase angle of said organ, tissue, meat, fish, fruit or vegetable;
then again placing said signal introduction electrodes on said superior and said inferior borders of said organ, tissue, meat, fish, fruit or vegetable;
again placing said signal detection electrodes on said opposite lateral borders of said organ, tissue, meat, fish, fruit or vegetable;
measuring and recording fourth values of said resistance and said reactance and calculating the phase angle of said organ, tissue, meat, fish, fruit or vegetable; and
comparing said first and second values to said third and fourth values to determine if the values are within a predetermined acceptable range of agreement denoting no further loss of said organ, tissue, meat, fish, fruit or vegetable vitality.

29. A method of organ, tissue, meat, fish, fruit or vegetable vitality assessment for transplantation, treatment, consumption, preservation, sale or transport of an organ, tissue, meat, fish, fruit or vegetable being assessed, comprising the steps of:
placing signal introduction electrodes on opposite lateral peripheral borders of said organ, tissue, meat, fish, fruit or vegetable upon harvesting of said organ tissue, meat, fish, fruit or vegetable;
placing signal detection electrodes at superior and inferior borders of said organ tissue, meat, fish, fruit or vegetable for a first part of an initial measurement upon said harvesting of said organ, tissue, meat, fish, fruit or vegetable;
measuring and recording first values of resistance and reactance of said organ, tissue, meat, fish, fruit or vegetable in said initial measurement;
then placing said signal introduction electrodes on said superior and said inferior borders of said organ, tissue, meat, fish, fruit or vegetable;
placing said signal detection electrodes on said opposite lateral borders of said organ, tissue, meat, fish, fruit or vegetable;
measuring and recording second values of said resistance and said reactance of said organ, tissue, meat, fish, fruit or vegetable; and
comparing said first and second values to normal values to determine if said organ, tissue, meat, fish, fruit or vegetable is acceptable or not for said transplantation, consumption, treatment or transportation effects.

30. A method according to claim 29, wherein:
if said organ, tissue, meat, fish, fruit or vegetable is acceptable, then prior to preserving, selling, implanting, consuming or further treatment said organ, tissue, meat, fish, fruit or vegetable, performing the following steps;
again placing said signal introduction electrodes on said opposite lateral peripheral borders of said organ, tissue, meat, fish, fruit or vegetable;
again placing said signal detection electrodes at said superior and said inferior borders of said organ, tissue, meat, fish, fruit or vegetable for a first part of an initial post-harvest (transport/treatment)/pre-implant, consumption, preservation, sale, treatment or transport measurement;
measuring and recording third values of resistance and reactance of said organ, tissue, meat, fish, fruit or vegetable in said initial post-harvest (transport, treatment)/pre-implant measurement;
then placing said signal introduction electrodes on said superior and said inferior borders of said organ, tissue, meat, fish, fruit or vegetable;
placing said signal detection electrodes on said opposite lateral borders of said organ, tissue, meat, fish, fruit or vegetable;
measuring and recording fourth values of said resistance and said reactance of said organ, tissue, meat, fish, fruit or vegetable; and
comparing said first and second values to said third and fourth values to determine if the values are within a predetermined acceptable range of agreement denoting no further loss of said organ, tissue, meat, fish, fruit or vegetable vitality.

31. A method according to claim 29, including:
harvesting said organ from a first species of biological entity; and
implanting said organ in a different species of biological entity.

32. A method according to claim 30, including:
harvesting said organ from a first species of biological entity; and
implanting said organ in a different species of biological entity.

33. A method according to claim 23, wherein:
said measured values of resistance and reactance and the calculation of phase angle changes will be compared to their previous values and considered in the rate of change either increase or decrease the assessment of fluid volumes, cellular architecture, freshness and vitality.

34. A method according to claim 23, including the steps of:
comparing and assessing homogeneity within heterogeneous populations based upon comparative values of calculated phase angles.

35. A method according to claim 23, wherein:
the severity, criticality or burden of an adverse condition is based upon said calculated phase angle value in that a higher value indicates a less severe, critical or burden of adversity and a lower value indicates a greater severity, criticality or burden of adversity.

36. A method according to claim 35 wherein:
the resources allocated or required to manage said adverse condition are based upon said calculated phase angle value in that the lower phase angle value entity requires greater resources than that of an entity with a greater phase angle value.

37. A method according to claim 36, wherein:
in those entities that experience a transient reduction of said phase angle value that does not fully return to the previous baseline phase angle value after apparent recovery that that entity is not fully recovered and may be predisposed to further adversity and require additional care and intervention.

38. A method according to claim 30, wherein:
the vitality of said organ will have different levels of vitality based upon its measured resistance, reactance and calculated phase angle which while it may not be optimal will be sufficient for its purpose and may further be used to classify its use for a corresponding recipient with the matching of a higher phase angle value to the recipient with a lower phase angle value, and conversely the matching of a organ with a lower phase angle value to a recipient of a higher phase angle value.

39. A method according to claim 23, wherein:
the freshness of a consumable biological foodstuff such as a meat, fish, fowl, fruit or vegetable is based upon said calculated phase angle value in which the higher said phase angle value as related to that value upon initial harvest or processing is compared to that level after transport or upon purchase or process for purchase.

40. A method according to claim 39, wherein:
said phase angle is used as a freshness indicator of said consumable biological foodstuff.
